(19) United States
(12) Patent Application Publication
(10) Pub. No.: US 2021/0252308 A1
(43) Pub. Date: Aug. 19, 2021

(54) DIRECT MEASUREMENT OF IMMUNE SYSTEM RADIOSENSITIVITY AND RADIOTHERAPY TREATMENT PLAN OPTIMIZATION

(71) Applicant: Indiana University Research and Technology Corporation, Indianapolis, IN (US)

(72) Inventors: Jian-Yue Jin, Indianapolis, IN (US); Wenhu Pi, Indianapolis, IN (US); Feng-Ming Kong, Indianapolis, IN (US); Weili Wang, Indianapolis, IN (US)

(73) Assignee: Indiana University Research and Technology Corporation, Indianapolis, IN (US)

(21) Appl. No.: 16/973,738

(22) PCT Filed: Jun. 10, 2019
(86) PCT No.: PCT/US2019/036278

§ 371 Date: Dec. 9, 2020

Related U.S. Application Data
(60) Provisional application No. 62/683,598, filed on Jun. 11, 2018.

(51) Int. Cl. A61N 5/10; C12N 13/00; G01N 33/50

(52) U.S. Cl. CPC A61N 5/1031; G01N 2800/52; G01N 33/5047; C12N 13/00

(57) ABSTRACT

Methods for directly measuring a patient's relative sensitivity to radiation therapy are provided. In particular, the methods provide for calculating a radiation sensitivity quotient for monocytes in culture. The methods can be incorporated into radiotherapy (RT) treatment planning systems, which are also provided. The methods can be used to optimize patient treatment plans, thereby developing patient-specific radiation treatment plans. Methods for treating a patient with radiotherapy with an optimized treatment plan are provided.

### STATEMENT OF GOVERNMENT SUPPORT

This invention was made with government support under CA142840 awarded by the National Institutes of Health. The Government has certain rights in the invention.

### BACKGROUND

Radiotherapy (RT) is a major modality for cancer treatment. Delivery of an optimal RT dose is vital for effective treatment; an insufficient RT dose will not provide adequate therapeutic effect, while excessive RT doses can unnecessarily damage healthy surrounding cells and tissues, including those of the immune system. Recently, it has been increasingly understood that the killing of normal immune cells of the immune system is a major cause affecting therapeutic outcome with radiotherapy. Effective RT requires a fine balance between maximally eradicating tumor cells while minimally killing the normal immune cells. And while technological advances and state-of-the-art instrumentation have enabled incredibly precise delivery of RT to tumor lesions with substantial reductions in injuries to normal tissues, including the immune system, patients are heterogeneous in their response to RT. Certain patients are more sensitive to radiation than others, resulting in differing optimal RT doses for different patients. The sensitive patient will have a smaller optimal dose than that for the radiation resistant patient.

While the physical interaction of radiation energy with living cells leaves little room for inter-individual variation in the initial yield of DNA damage, variation between patients results from the inter-individual variation in downstream processes in how such damage is recognized, repaired, or resolved. In theory, radiosensitivity can be predicted in individuals with genetic determinants of radiosensitivity, and many studies have reported potential genetic determinants. However, to date, the studies of genetic determinants have been controversial.

Functional assays for cellular radiosensitivity represent a strategy to identify patients with potential radiosensitivity. Functional assays involving surrogate measures of normal tissue radiosensitivity through ex vivo assays on lymphocytes or fibroblasts have been proposed. Functional assays on lymphocytes directly measure the radiosesnitivities of the immune system, while functional assays on fibroblasts measure the radiosesnitivities of other normal cells. The radiosesnitivities of lymphocytes, other normal tissues and tumor have some similarities because they share some common genetic origin; however, they are not completely the same.

Since radiation-induced chromosomal damage leading to cell death or loss of cellular reproductive capacity is largely considered the primary mechanism by which normal tissues suffer injury during radiotherapy, chromosomal aberrations and clonogenic survival represent common endpoints in the prediction of cellular radiosensitivity. However, measuring chromosomal aberration is not a direct measure of cell death. The two endpoints are not the same. In addition, it is difficult to clone lymphocytes, and conventional clonogenic assays cannot be performed for lymphocytes. Although a limited dilution clonogenic survival assay has been proposed, it requires feeder cells to be added to the cell culture plates, and testing requires a serial dilution. This procedure is also time and labor intensive, and generally requires cell transformation which can interfere with the inherent radiosensitivity of cells.

### SUMMARY

In a first example (“Example 1”), described herein is a method comprising irradiating one or more cultures of adherent monocytes derived from a single test sample of peripheral blood with a preselected radiation dose, wherein each of the one or more cultures of adherent monocytes is irradiated with a different preselected radiation dose; removing, at a preselected time point following irradiation, a fraction of the irradiated adherent monocytes from each of the one or more cultures of adherent monocytes; counting a number of viable cells in each fraction; and calculating, for each fraction, a radiation sensitivity quotient by calculating a difference in radiation dose between the fraction and a control that results in a same number of viable cells.

In another example (“Example 2”) further to Example 1, the method further comprises removing at least one additional fraction of the irradiated adherent monocytes from each of the one or more cultures of adherent monocytes, wherein each of the at least one additional fractions is removed at a second preselected time point; counting a number of viable cells in each of the at least one additional fractions; and calculating, for each of the at least one additional fractions, a radiation sensitivity quotient. In another example (“Example 3”) further to Example 2, the steps of Example 2 can be repeated at least once at at least one additional preselected time point.

In another example (“Example 4”) further to any one of Examples 1-3, at least three cultures of adherent monocytes are each irradiated with a different preselected radiation dose.

In another example (“Example 5”) further to any one of Examples 1-3, four cultures of adherent monocytes are each irradiated with a different preselected radiation dose.

In another example (“Example 6”) further to any one of Examples 1-4, a first culture of adherent monocytes is irradiated with a radiation dose of 2 Gy, a second culture of adherent monocytes is irradiated with a radiation dose of 4 Gy, a third culture of adherent monocytes is irradiated with a radiation dose of 8 Gy, and a fourth culture of adherent monocytes is irradiated with a radiation dose of 12 Gy.

In another example (“Example 7”) further to any one of Examples 1-6, the method further comprises generating a survival-dose curve for a given time point, wherein the difference in radiation between the fraction and a control that results in a same number of viable cells, and thus the radiation sensitivity quotient, is calculated from the survival-dose curve and a control survival dose curve.

In another example (“Example 8”) further to any one of Examples 1-7, the method further comprises comprising calculating an average radiation sensitivity quotient, wherein the average radiation sensitivity quotient is calculated from two or more calculated sensitivity quotients.

In another example (“Example 9”) further to any one of Examples 1-8, the single test sample of peripheral blood is from a patient undergoing radiotherapy or is to undergo radiotherapy.

In another example (“Example 10”) further to any one of Examples 1-9, the method further comprises establishing one or more cultures of adherent monocytes from a single test sample of peripheral blood.

In another example (“Example 11”) further to Example 10, the method further comprises collecting the sample of peripheral blood.

In another example described herein (“Example 12”) is a method for treating a patient, the method comprising performing a method according to any one of Examples 1-11 and administering an optimized radiotherapy dose to the patient, wherein the optimized radiotherapy dose is determined by reducing a standard radiotherapy dose by the radiation sensitivity quotient.

In another example described herein (“Example 13”) is a method for treating a patient, the method comprising obtaining a radiation sensitivity quotient determined by the methods according to any one of Examples 1-11, and administering an optimized radiotherapy dose to the patient, wherein the optimized radiotherapy dose is determined by reducing a standard radiotherapy dose by the radiation sensitivity quotient.

In another example described herein (“Example 14”) is a method for generating a patient-specific radiotherapy treatment plan, the method comprising performing a method according to any one of Examples 1-11, reducing a standard radiotherapy dose in a reference radiotherapy treatment plan by the radiation sensitivity quotient.

In another example described herein (“Example 15”) is a method for generating a patient-specific radiotherapy treatment plan, the method comprising obtaining a radiation sensitivity quotient determined by the methods according to any one of Examples 1-11, and reducing a standard radiotherapy dose in a reference radiotherapy treatment plan by the radiation sensitivity quotient.

### BRIEF DESCRIPTION OF THE DRAWINGS

FIG. 1 is a flowchart illustrating a method according to one embodiment.

FIG. 2 is a flowchart illustrating a method according to one embodiment.

FIG. 3 is a flowchart illustrating a method according to one embodiment.

FIG. 4 is a block diagram illustrating a system formed in accordance with one embodiment that may be used to carry out the methods described herein.

FIGS. 5A-5C are line graphs illustrating the direct measurement of radiosensitivity of cultured monocytes from a control (normal) patient and a radiosensitive patient according to one embodiment. The number of viable cells following administration of the indicated radiation dose at 5 days (FIG. 5A), 7 days (FIG. 5B), and 10 days (FIG. 5C) after radiation is indicated.

While the disclosed subject matter is amenable to various modifications and alternative forms, specific embodiments are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

### DETAILED DESCRIPTION

Certain embodiments described herein provide methods for directly measuring the radiosensitivity of the immune system of a patient undergoing or about to undergo radiotherapy (RT). In some embodiments, the methods described herein can be used to optimize patient treatment plans. Also provided are methods for treating a patient with RT with an optimized treatment plan developed according to the methods described herein.

As discussed below in more detail, portions of these methods can be implemented using a processor executing software stored in a tangible, non-transitory storage medium. For example, the software could be stored in the long-term memory (e.g., solid state memory) in a radiotherapy system, executed by the processor(s) in the radiotherapy system. In other embodiments, the software could be stored in a separate system.

Functional assays for cellular radiosensitivity represent a strategy to identify patients with potential radiosensitivity. Many of the functional assays proposed to date involve surrogate measures of normal tissue radio sensitivity through ex vivo assays on lymphocytes or fibroblasts. It had been considered that lymphocytes, other normal tissues and even tumor share some common genetic origin, so that the patient with radiosensitive lymphocytes would also have other normal tissues and tumors that are radiosensitive. However, studies have demonstrated that the radiosensitivity of lymphocytes is not the same as other normal tissues and tumors. Functional assays on lymphocytes can only directly measure the radiosesnitivities of the immune system, while functional assays on fibroblasts measures the radiosesnitivities of the particular fibroblasts. To date, the use of functional assays for cellular radiosensitivity to predict normal tissue toxicity has been controversial.

The immune system has largely not been considered as an organ at risk for RT toxicity. However, studies by the inventors have indicated that the radiation dose delivered to the immune system of a patient is a key predictor for success of treatment and overall survival. Thus, functional assays on lymphocytes are ideal approaches to directly measure the radiosesnitivities of immune system and guide optimization of the radiation dose.

Historically, fibroblast cells have been used to predict radiosensitivity in patients due to their high adhesion. However, assays conducted with fibroblasts were demonstrated to be weakly associated with a patient's acute radiosensitivity, with fibroblasts being demonstrated to be resistant to radiation.

Irradiated lymphocytes, which include T cells and B cells, have also been used predict a patient's radiosensitivity. However, lymphocytes are difficult to clone. Conventional clonogenic assays cannot be performed for lymphocytes. Although a limited dilution clonogenic survival assay has been proposed, it requires feeder cells to be added to the cell culture plates, and testing requires a serial dilution. This procedure is time and labor intensive and generally requires cell transformation, which can interfere with the inherent radiosensitivity of cells. Recently, 2D and 3D culture systems have been used for peripheral blood lymphocyte culture. However, these 2D and 3D matrix plates, as well as the feeder layers, are expensive and labor intensive. Some studies have used flow cytometry to analyze irradiated lymphocytes and found the analysis to be comparable to the clonogenic assay. Although flow cytometry (FACS) is easier and faster, it requires specific antibody staining Other studies have assayed radiation sensitivity using radiation damages such as chromosomal aberrations and double-strand breaks. The γH2AX foci assay is frequently used to detect double stranded breaks, and the assay remains complicated, as the number of γH2AX foci has to be counted. The quantification is generally done manually, resulting in a time-consuming effort prone to human error. Efforts to automate detection of double-strand breaks rely on image analysis and require complicated computing.

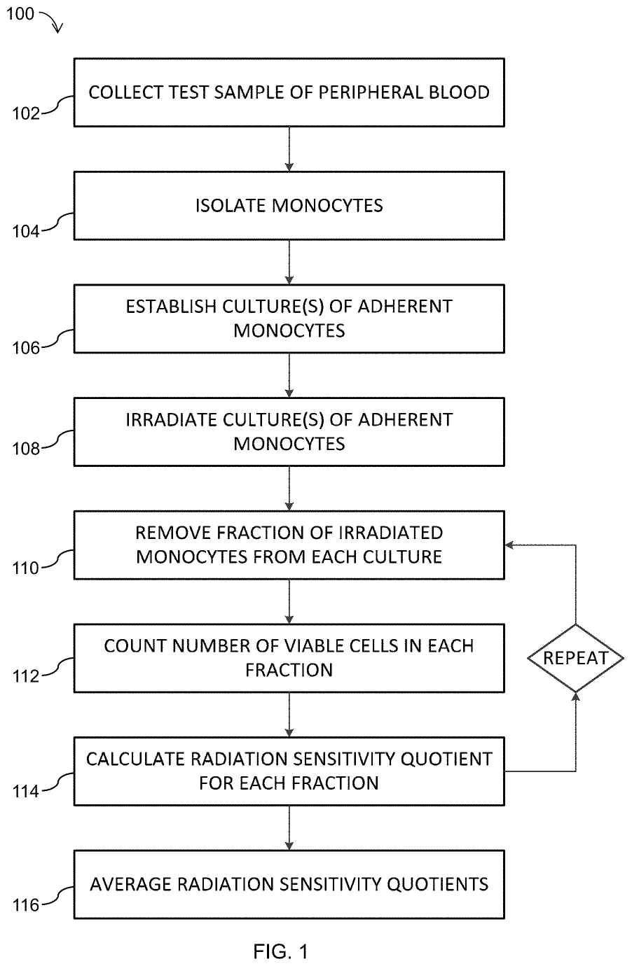

Certain embodiments provide methods for directly measuring a patient's radiosensitivity. In some embodiments, the methods measure the radiosensitivity of a patient's immune system. FIG. 1 is a flowchart representing an embodiment of the methods described herein. The method 100 comprises collecting a test sample of peripheral blood 102, isolating monocytes therefrom 104, establishing at least one cell culture of adherent monocytes 106, irradiating each of the cultures of adherent monocytes 108, removing a fraction of the irradiated monocytes from each of the cultures 110, counting a number of viable cells in each fraction 112, calculating a radiation sensitivity quotient for each fraction 114, optionally repeating steps 110-114 at least once, and averaging the calculated radiation sensitivity quotients 116.

The test sample of peripheral blood is collected from a patient undergoing or about to undergo RT. Test samples of peripheral blood can be collected by standard methods, such as venipuncture sampling. Monocytes are then isolated from the test sample. Methods for isolating monocytes from peripheral blood samples are well known in the art, and include for example, fluorescence activated cell sorting (FACS), magnetic activated cell sorting (MACS), density gradient centrifugation and double density gradient centrifugation, plastic/glass adherence isolation, and bipolar tetrameric antibody-based separation.

Following isolation of monocytes from the test sample of peripheral blood, the isolated monocytes are plated onto at least one cell culture dish. In some embodiments, approximately 2.0×10⁶ to 1.0×10⁷ cells are established on each culture dish. Methods and media suitable for establishing monocytes in culture are well known in the art. In some embodiments, isolated monocytes are cultured with RPMI 1640 media containing 10% FBS and 1% P/S. After a sufficient incubation period, non-adherent cells are removed, leaving a cell culture of adherent monocytes. In some embodiments, the monocytes are cultured overnight prior to removing non-adherent cells. In some embodiments, all cultures of adherent monocytes are established from a single test sample of peripheral blood.

Once the culture(s) of adherent monocytes are established, the cell culture(s) of adherent monocytes is(are) irradiated with a preselected radiation dose. When two or more cultures of adherent monocytes are to be irradiated, each culture is irradiated with a different preselected radiation dose. In some embodiments, at least three cultures of adherent monocytes are established, and each culture is irradiated with a different radiation dose. In some embodiments, four cultures of adherent monocytes are established, and each culture is irradiated with a different radiation dose.

In some embodiments, the preselected radiation dose is a radiation dose typically encountered by a patient undergoing radiotherapy. In some embodiments, the preselected radiation dose is a radiation dose typically encountered by a patient during a single fraction, over one day, over one week, or over an entire course of treatment. In some embodiments, the preselected radiation dose is a radiation dose that is higher than that typically encountered by a patient undergoing radiotherapy over a given time course. This allows for the investigation of the upper limits of radiation that may be suitable for a particular patient.

In some embodiments, the preselected radiation dose is selected from 0.5 Gy to 60 Gy. In some embodiments, the preselected radiation dose is selected from 0.5 Gy, 1 Gy, 2 Gy, 4 Gy, 8 Gy, 12 Gy, 16 Gy, and 20 Gy.

In some embodiments, four cultures of adherent monocytes are established, with each of the four cultures being irradiated with a different preselected radiation dose. In some embodiments, a first culture of adherent monocytes is irradiated with a radiation dose of 2 Gy, a second culture of adherent monocytes is irradiated with a radiation dose of 4 Gy, a third culture of adherent monocytes is irradiated with a radiation dose of 8 Gy, and a fourth culture of adherent monocytes is irradiated with a radiation dose of 12 Gy.

Following irradiation of the culture(s) of adherent monocytes, the irradiated cultures of adherent monocytes are incubated under appropriate cell culture conditions for a sufficient period of time. At a preselected time point following irradiation, a fraction of the irradiated adherent monocytes from each the cultures of adherent monocytes is collected. In some embodiments, a fraction of irradiated monocytes is removed from the culture dish(es) at regular time intervals. In some embodiments, the fraction of cells removed at each time point remain approximately constant, and the fractions are collected and removed from the culture dish(es) at regular intervals. For example, in some embodiments, approximately 10% of the cells in a culture dish are removed every day for 10 consecutive days following irradiation. In some embodiments, approximately 10% of the cells in a culture dish are removed every second day following irradiation. In other embodiments, 20% of the cells in a culture dish are collected and removed every two or three days following irradiation.

Once a fraction of irradiated monocytes is collected and removed from a culture of adherent monocytes, the number of viable cells in the fraction is counted. A viable cell count allows for the identification of a number of actively growing/dividing cells in the fraction, and thus serves to provide a direct measurement of the effect of irradiation by the preselected radiation dose. The number of viable cells in each fraction can be counted using a hemocytometer or can be counted using an automated cell counter. In some embodiments, the number of viable cells in each fraction is counted using an automated cell counter. Many automated viable cell counters are available, and include counters from companies such as Bio-Rad®, Nexcelom Bioscience®, MilliporeSigma®, Beckman Coulter®, Eppendorf®, Logos Biosystems®, Olympus®, and Thermo Fisher Scientific®.

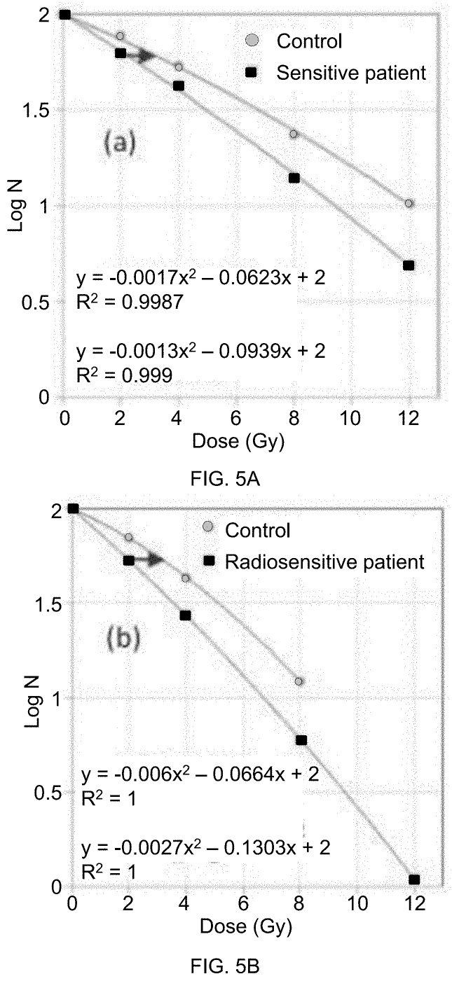

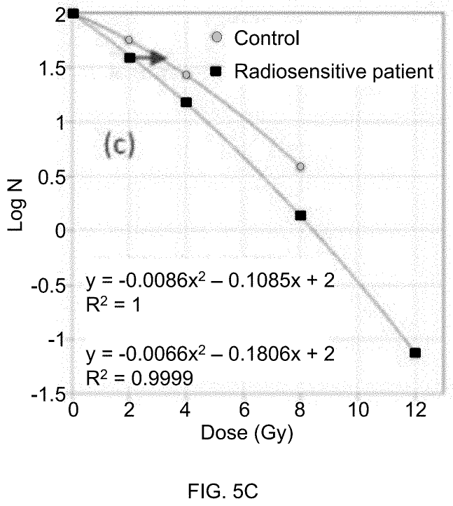

The viable cell count for each fraction is used to calculate a radiation sensitivity quotient for each fraction. In some embodiments, the radiation sensitivity quotient represents the difference in radiation required to result in an identical viable cell count in each fraction and a corresponding control. The controls are from one or more subjects having a known radiosensitivity, and are matched to each fraction for fraction size (i.e., number of cells), radiation dose, and time point of collection. In some embodiments, the radiation sensitivity quotient is calculated by determining the difference in radiation between a test sample fraction and a control sample fraction that results in an identical viable cell count. For example, FIG. 5A presents a dose-survival curve for fractions taken 5 days following irradiation. At a radiation dose of 2 Gy, the viable cell count for the sensitive patient (i.e., the test sample) is approximately 1.8 log N. The radiation sensitivity quotient is calculated as the difference in radiation required to give that same viable cell count of 1.8 log N. If FIG. 5A, this difference in radiation (i.e., the radiation sensitivity quotient) is illustrated by the arrow. By extrapolation, a dose of approximately 3 Gy would result in a viable cell count of 1.8 log N in the control patient. Indeed, the radiation sensitivity quotient was calculated to be 44% (see Example 1).

In certain embodiments, the number of viable cells counted in fractions taken from different culture dishes (i.e., irradiate at different radiation doses) but at the same time point following irradiation are used to generate a survival-dose curve. For example, the number of viable cells counted in fractions collected from culture dishes 5 days following irradiation with 2 Gy, 4 Gy, 8 Gy, or 12 Gy can be fitted with a linear quadratic model and plotted as a survival-dose curve (see FIGS. 5A-5C). The number of viable cells in a control sample can be similarly determined and plotted. The difference in viable cells at a given radiation dose can be determined from the resulting survival-dose curves.

In some embodiments, a radiation sensitivity quotient is calculated at more than one radiation dose. In the example provided above, a radiation sensitivity quotient can be calculated from the survival-dose curves at each of 2 Gy, 4 Gy, 8 Gy, and 12 Gy.

In some embodiments, a radiation sensitivity quotient is calculated for one or more radiation doses at more than one time point following irradiation. For example, if fractions from culture dishes irradiated with 2 Gy, 4 Gy, 8 Gy, and 12 Gy are collected on days 5, 7, and 10 following irradiation, a radiation sensitivity quotient for one or more of these radiation doses can be calculated for each time point.

In some embodiments, an average radiation sensitivity quotient is determined. In some embodiments, an average radiation sensitivity quotient is calculated by determining the average radiation sensitivity quotient for two or more different radiation doses from the same time point (e.g., average of radiation sensitivity quotients calculated for each dose of 2 Gy, 4 Gy, 8 Gy, and 12 Gy, 5 days following irradiation). In some embodiments, an average radiation sensitivity quotient is calculated by determining the average radiation sensitivity quotient for the same radiation dose from different days (e.g., average of radiation sensitivity quotients calculated for a dose of 2 Gy at 5, 7, and 10 days following irradiation). In some embodiments, an average radiation sensitivity quotient is calculated by determining the average radiation sensitivity quotient for two or more different radiation doses from different days (e.g., average of radiation sensitivity quotients calculated for each dose of 2 Gy, 4 Gy, 8 Gy, and 12 Gy, at each of 5, 7, and 10 days following irradiation).

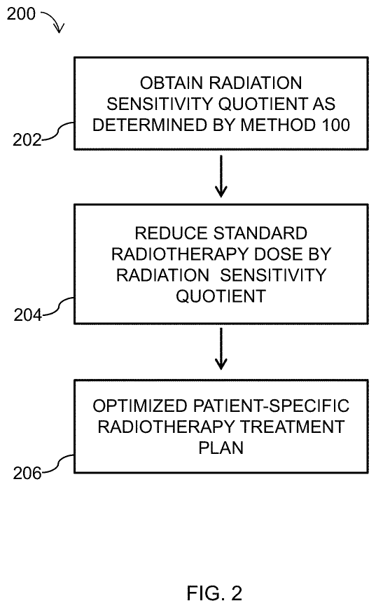

Some embodiments provide methods for optimizing a radiotherapy treatment plan for a patient undergoing radiotherapy or scheduled to undergo radiotherapy, thus generating a patient-specific radiotherapy treatment plan. FIG. 2 provides a graphical representation of such methods 200. In some embodiments, the radiation sensitivity quotient or average radiation sensitivity quotient determined according to methods described herein 202 (e.g., according to method 100) can be used to generate a patient-specific radiotherapy treatment plan for a patient 206.

In some embodiments, generating a patient-specific radiotherapy treatment plan comprises calculating a radiation sensitivity quotient (or average radiation sensitivity quotient) according to the present disclosure 202, and reducing a standard radiotherapy dose from a reference radiotherapy treatment plan by the radiation sensitivity quotient 204 to generate an optimized patient-specific radiotherapy treatment plan 206. The reference radiotherapy treatment plan can be a plan according to the standard of care, or standard radiotherapy dose prescription for a particular cancer. For example, where a standard radiotherapy dose prescription calls for 20 Gy to be delivered in 10, 2 Gy fractions, the total and daily radiation doses of 20 Gy and 2 Gy, respectively, are reduced by the calculated radiation sensitivity quotient. In this example, where a radiation sensitivity quotient of 40% is calculated for a radiosensitive patient, the optimized radiotherapy treatment plan for the patient would be 12 Gy deliver in 10, 1.2 Gy fractions (i.e., a 40% reduction relative to the standard radiotherapy dose prescription).

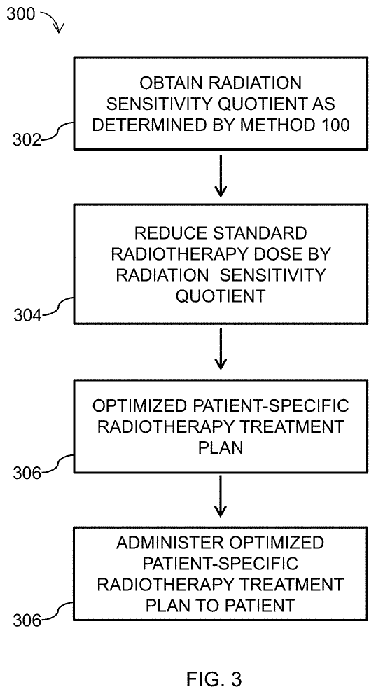

Some embodiments provide methods for treating a patient with radiotherapy. FIG. 3 provides a graphical representation of such methods 300. In some embodiments, a patient-specific radiotherapy dose 306 is determined by reducing a standard radiotherapy dose by the patient's radiation sensitivity quotient 304 or average radiation sensitivity quotient (obtained by the methods provided herein, e.g., method 100), and the patient-specific radiotherapy dose is delivered to the patient 306. The standard radiotherapy dose can be a standard radiotherapy dose prescription for a particular cancer. In some embodiments, a patient-specific radiotherapy treatment plan can be determined according to the methods described herein (e.g., method 200), and the patient is treated according to the patient-specific radiotherapy treatment plan.

In some embodiments, the methods for calculating the radiation sensitivity quotient or average radiation sensitivity quotient are carried out on one or more suitably programmed computers. In some aspects, methods for calculating the radiation sensitivity quotient or average radiation sensitivity quotient for a patient and generating a patient-specific radiotherapy treatment plan are carried out on a radiotherapy system.

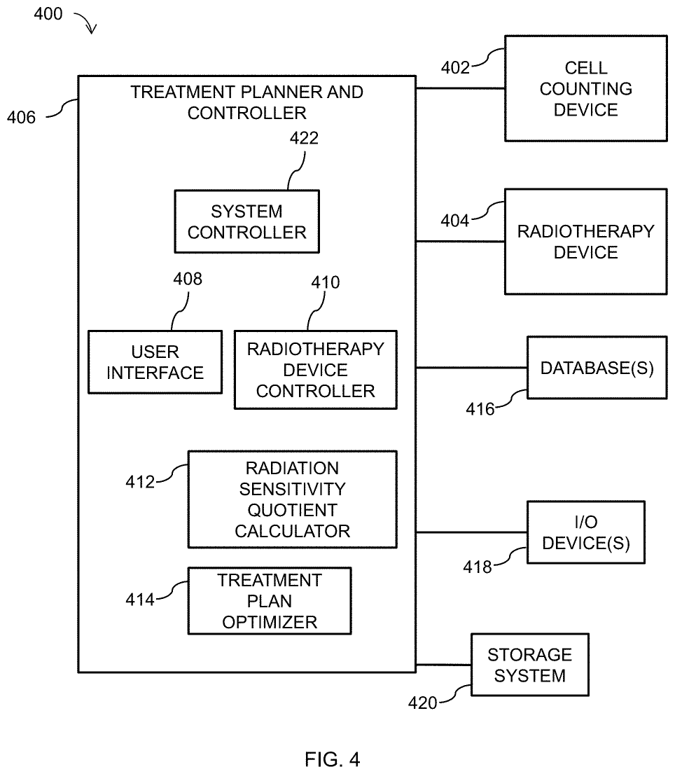

FIG. 4 illustrates a radiotherapy system 400 formed in accordance with an embodiment that can be used to carry out the methods disclosed and described herein. For example, the system 400 can be used to carry out the methods, including methods 100 (FIG. 1), 200 (FIGS. 2), and 300 (FIG. 3). In some embodiments, the methods can be automated by the system 400. In some embodiments, certain steps of the methods can be automated by the system 400 while others may be performed manually or otherwise require user interaction. In some embodiments, the user provides an initial treatment plan for a patient to the system 400, or otherwise causes an initial treatment plan to be provided to the system 400, and the system 400 automatically generates a patient-specific radiotherapy treatment plan.

In some embodiments, radiotherapy system 400 is an integrated standalone system that is located at one site. In other embodiments, one or more components of the system are located remotely with respect to each other. For example, in some embodiments, the radiation sensitivity quotient calculator 412, treatment plan optimizer 414, database(s) 416, and storage system 420 may be implemented in multiple instances, distributed across multiple computing devices, instantiated within multiple virtual machines, and the like.

As depicted, the radiotherapy system 400 comprises a cell counting device 402, a radiotherapy device 404, a treatment controller 406 comprising a user interface 408, a radiotherapy device controller 410, a radiation sensitivity quotient calculator 412, a treatment plan optimizer 414, one or more databases 416; one or more input/output (I/O) devices 418, and a storage system 420. In some embodiments, the database 416 provides past or proposed (i.e., initial) RT treatment plans and/or patient records to the treatment controller 406.

FIG. 4 provides a block diagram of a treatment controller 406 according to one embodiment. In some embodiments, the treatment controller 406 can calculate the radiosensitivity quotient for a given patient and/or control a radiotherapy device according to patient-specific treatment plan. In some embodiments, the treatment controller 406 comprises a system controller 422, a user interface 408, a radiotherapy (RT) device controller 410, a radiation sensitivity quotient calculator 412, and a treatment plan optimizer 414. The system controller 422 is communicatively coupled to the user interface 408 and/or the radiotherapy device 404. In some embodiments, the system control 422 comprises one or more processors/modules to calculate a radiosensitivity quotient and, optionally, optimize a treatment plans in accordance with the methods described herein. For example, in some embodiments, the system control 422 includes one or more modules, each module being configured to execute a set of instructions that are stored in one or more storage elements (e.g., instructions stored on a tangible and/or non-transitory computer readable storage medium) to calculate radiosensitivity quotients and, optionally, optimize the treatment plan. In some embodiments the set of instructions includes various commands that instruct the system controller 520 as a processing machine to perform specific operations such as the processes and methods described herein.

As illustrated, the treatment controller 406 comprises a plurality of modules or submodules that control operation of the system controller 422. In some embodiments, the treatment controller 406 includes modules 410, 412, and 414, which are connected to or form a part of the system controller 422, and are connected to a storage system 420 and one or more databases 416. The storage system 420 and databases 416 can communicate with at least some of the modules 410, 412, 414, and system controller 422. In some embodiments, the modules comprise a radiotherapy device controller 410, a radiation sensitivity quotient calculator 412, and a treatment plan optimizer 414. In some embodiments, the radiotherapy system 400 comprises additional modules or sub-modules, configures to perform the operations and methods described herein.

In some embodiments, the radiotherapy system comprises a cell counting device 402. The cell counting device 402 is configured to receive one or more fractions of irradiated, adherent monocytes, and for each fraction, count the number of viable cells. In some embodiments, the cell counting device 402 is configured to provide the counted number of viable cells in each fraction to the radiation sensitivity quotient calculator 412. The cell counting device can be any automated cell counting device capable of counting viable cells in a sample.

The radiation sensitivity quotient calculator 412 is configured to receive viable cell count data and/or viable cell control data from the cell counting device 402 or an outside source via the I/O device 418, and to calculate the radiation sensitivity quotient for a patient according to the methods described herein.

The treatment plan optimizer 414 is configured to optimize a treatment plan and generate a patient-specific radiotherapy treatment plan according to the methods described herein.

The radiotherapy device controller 410 is configured to receive a patient-specific radiotherapy treatment plan from treatment plan optimizer 414, and to control radiotherapy device 404. The radiotherapy device controller 510 is configured to cause the radiotherapy device 404 to administer a radiotherapy according to a patient-specific radiotherapy treatment plan.

By way of example, the treatment controller 406 can be or include a desktop computer, a laptop computer, a notebook computer, a tablet computer, a smart phone, and the like. In some embodiments, the user interface 408 includes hardware, firmware, software, or a combination thereof that enables a user to directly or indirectly control operation of the system controller 422 and the various other modules and/or sub-modules. In some embodiments, the radiotherapy system 400 comprises an input/output (I/O) device 416, such as a keyboard, display printer, universal serial bus (USB) port, a speaker, pointer device, trackball, button, switch, touch screen, and the like.

In some embodiments, the radiotherapy system 400 displays a standard radiotherapy dose prescription and the resulting patient-specific radiotherapy treatment plan on an I/O device 418 that is a display. In other embodiments, the radiotherapy system 400 is configured to deliver a patient-specific radiotherapy treatment plan to a printer, an email address, or other output.

In some embodiments, the radiotherapy system 400 comprises only those components necessary to generate a patient-specific radiotherapy treatment plan. For example, in some embodiments, the radiotherapy device 404 and the radiotherapy device controller 410 are excluded. Thus, in some embodiments, a radiotherapy treatment controller is provided. The radiotherapy treatment controller can be the same as the treatment controller 406 described above.

In some embodiments, a radiotherapy system 400 also includes one or more imaging modalities suitable for acquiring images of areas of interest, such as a target irradiation area within a patient. Suitable imaging modalities include, for example, computed tomography (CT) scanners, positron emission tomography (PET) scanners, magnetic resonance (MR) scanners, single photon emission computed tomography (SPECT) scanners, and the like. In some embodiments, the images acquired by the imaging modalities are three-dimensional images. In other embodiments, the images are two-dimensional. In certain embodiments, three-dimensional images include a stack of two dimensional images (i.e., slices).

As used herein, the terms “module,” “system,” and “system controller” can refer to a hardware and/or software system and circuitry that operates to perform one or more functions. A module, system, or system controller may include a computer processor, controller, or other logic-based device that performs operations based on instructions stored on a tangible and non-transitory computer readable storage medium, such as a computer memory. Alternatively, a module, system, or system controller can include a hard-wired device that performs operations based on hard-wired logic and circuitry. The module, system, or system controller depicted by FIG. 4 can represent the hardware and circuitry that operates based on software or hardwired instructions, the software that directs hardware to perform the operations, or a combination thereof. The module, system, or system controller can include or represent hardware circuits or circuitry that include and/or are connected with one or more processors, such as one or more computer microprocessors.

As used herein, the terms “software” and “firmware” are interchangeable, and include any computer program stored in memory for execution by a computer, including Random Access Memory (RAM), Read Only Memory (ROM), Electronically Erasable Programmable Read Only Memory (EEPROM), non-volatile RAM (NVRAM), flash memory, optical or holographic media, magnetic cassettes, magnetic tape, magnetic disk storage or other magnetic storage devices, data transmissions, or any other medium that can be used to store information and can be accessed by a computing device. The above memory types are representative only, and are thus not limiting as to the types of memory usable for storage of a computer program.

In some embodiments, a processing unit, processor, module, or computing system that is “configured to” perform a task or operation can be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). A general purpose computer (which may become “configured to” perform the task or operation if appropriately programmed) is not “configured to” perform a task or operation unless or until specifically programmed or structurally modified to perform the task or operation.

In some embodiments, the memory stores computer-executable instructions for causing the system controller 422 to implement aspects of embodiments of system components discussed herein and/or to perform aspects of embodiments of methods and procedures discussed herein. Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, elements of the radiotherapy system 400, such as the treatment controller 406 and modules or sub-modules thereof, database(s) 416, I/O device(s) 418, storage system 420, and radiotherapy device 404 are communicatively coupled by one or more communication links. In some embodiments, the one or more communication links can be, or include, a wired communication link such as a USB link, a proprietary wired protocol, and the like. The one or more communication links can be, or include, a wireless communication link such as a short-range radio link, such as Bluetooth IEEE 802.11, a proprietary wireless protocol, and the like.

The term “communication link” can refer to an ability to communicate some type of information in at least one direction between at least two elements of a computer system, and should not be understood to be limited to a direct, persistent, or otherwise limited communication channel That is, according to some embodiments, the communication link may be a persistent communication link, an intermittent communication link, an ad-hoc communication link, and the like. The communication link can refer to direct communications or indirect communications between the radiotherapy device controller 410 and the radiotherapy device 404, between the database(s) 416 and the radiation sensitivity quotient calculator 412, between the user interface 408 and the treatment plan optimizer 414, or any other combination of the elements of the radiotherapy system 400, wherein the indirect communication occurs via at least one other device (e.g., a repeater, router, hub, and/or the like). The communication link can facilitate unidirectional and/or bi-directional communication between the various elements of the radiotherapy system 400. In some embodiments, the communication link is, includes, or is included in a wired network, a wireless network, or a combination of wired and wireless networks. Illustrative networks include any number of different types of communication networks such as, a short messaging service (SMS), a local area network (LAN), a wireless LAN (WLAN), a wide area network (WAN), the Internet, a peer-to-peer (P2P) network, or other suitable networks. The network may include a combination of multiple networks. In some embodiments, for example, the radiotherapy system is accessible via the Internet (e.g., the radiotherapy system may facilitate a web-based RT treatment plan optimization/selection service), and a user may transmit one or more possible RT treatment plans to the radiotherapy system to optimize/select an adjusted RT treatment plan (i.e., a patient-specific radiotherapy treatment plan).

In some embodiments, the system controller 422 causes the radiation sensitivity quotient calculator 412to access the database 416 and/or I/O device 418 to obtain one or more initial RT treatment plans and/or viable cell control data via a communication link. Intermediary RT treatment plan data and/or viable cell control data from the database(s) 416 can be web-based, cloud based, or local. In some embodiments the initial RT treatment plan data, viable cell control data, and/or the databases 416 are retrieved from a third party, produced by the user, or some combination thereof. The databases 416 can be any collection of information providing, for example, information regarding common RT treatment plans (i.e., standard radiotherapy dose prescription, patient data, viable cell control data, and the like).

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1

In one example of the embodiments described herein, it is demonstrated that radiation sensitivity can be directly measured in cultured monocytes from peripheral blood.

The study described in this example found that monocytes from a patient who developed early esophagitis in the middle of treatment demonstrated much higher sensitivity than the control patient (>40% higher sensitivity). It is hypothesized that patients who develop esophagitis after a full dose of radiation treatment tend to be more radiosensitive. Previous studies have indicated that these patients had significantly worse local progression-free survival, progression-free survival, and overall survival. It is believed that sever immune toxicity due to the patient's radiosensitivity has caused the poor tumor control and survival. The present study aimed to directly measure the radiosensitivity of patient's. The measured radiosensitivity can be used to determine individualized optimal doses, and consequently improve survival.

Radiosensitivity of cultured monocytes derived from a control (normal) patient and a patient demonstrated to be radiosensitive was measured. The number of viable cells remaining following irradiation with either 2 Gy, 4 Gy, 8 Gy, or 12 Gy was counted at 5 days (FIG. 5A), 7 days (FIG. 5B), and 10 days (FIG. 5C) after radiation. The log of the normalized viable cell number (log N) vs. radiation dose was plotted and fitted with a linear quadratic model. This allows for the quantification of the radiosensitivity.

The model was used to calculate the difference in radiosensitivity as the difference in radiation dose relative to the control that results in the same number of viable cells at a dose of 2 Gy for the sensitive patient. The difference is denoted by the arrow in FIGS. 5A-5C. The difference in radiosensitivity, or the radiation sensitivity quotient, was 44%, 42%, and 45% at 5, 7, and 10 days following radiation, respectively.

### Materials and Methods

Peripheral blood samples were collected from a radiation-sensitive patient and a healthy volunteer. Blood samples were centrifuged using a Ficoll-Paque solution at 500× g in order to isolate monocytes from the peripheral blood samples. The isolated monocytes were washed with PBS, and cultured with RPMI 1640 medium containing 10% FBS and 1% P/S overnight. The following day, non-adherent cells were removed.

Monocytes were split into separate cultures to allow for radiation with 0 Gy (control), 1 Gy, 2 Gy, 4 Gy, 8 Gy, or 12 Gy. Approximately 2.0×10⁶-1.0×10⁶ cells were plated to establish each culture. Cells were then irradiated. Following irradiation, 10% of the cells were collected and removed from each culture. Collected fractions were washed with PBS and then suspended in 50 μl PBS. Each 50 μl cell sample was combined with 50 μl of 0.2% trypan blue (final concentration of 0.1% trypan blue). Viable cells were then counted using an automated cell counter (Nexcelom) according to the manufacturer's instructions.

Cell survival-dose curves were then plotted and analyzed as described above.

1. A method comprising:
irradiating one or more cultures of adherent monocytes derived from a single test sample of peripheral blood with a preselected radiation dose, wherein each of the one or more cultures of adherent monocytes is irradiated with a different preselected radiation dose, removing, at a preselected time point following irradiation, a fraction of the irradiated adherent monocytes from each of the one or more cultures of adherent monocytes, counting a number of viable cells in each fraction, and calculating, for each fraction, a radiation sensitivity quotient by calculating a difference in radiation between the fraction and a control that results in a same number of viable cells.

2. The method of claim 1, further comprising:
removing at least one additional fraction of the irradiated adherent monocytes from each of the one or more cultures of adherent monocytes, wherein each of the at least one additional fractions is removed at a second preselected time point,
counting a number of viable cells in each of the at least one additional fractions, and calculating, for each of the at least one additional fractions, a radiation sensitivity quotient.

3. The method of claim 2, further comprising:
repeating the method of claim 2 at least once at at least one additional preselected time point.

4. The method of claim 1, wherein at least three cultures of adherent monocytes are each irradiated with a different preselected radiation dose.

5. The method of claim 1, wherein four cultures of adherent monocytes are each irradiated with a different preselected radiation dose.

6. The method of claim 1, wherein a first culture of adherent monocytes is irradiated with a radiation dose of 2 Gy, a second culture of adherent monocytes is irradiated with a radiation dose of 4 Gy, a third culture of adherent monocytes is irradiated with a radiation dose of 8 Gy, and a fourth culture of adherent monocytes is irradiated with a radiation dose of 12 Gy.

7. The method of claim 1, further comprising generating a survival-dose curve for a given time point, wherein the difference in radiation between the fraction and a control that results in a same number of viable cells, and thus the radiation sensitivity quotient, is calculated from the survival-dose curve and a control survival dose curve.

8. The method of claim 1, further comprising calculating an average radiation sensitivity quotient, wherein the average radiation sensitivity quotient is calculated from two or more calculated sensitivity quotients.

9. The method of claim 1, wherein the single test sample of peripheral blood is from a patient undergoing radiotherapy or is to undergo radiotherapy.

10. The method of any claim 1, further comprising establishing one or more cultures of adherent monocytes from a single test sample of peripheral blood.

11. The method of claim 10, further comprising collecting the test sample of peripheral blood.

12. A method for treating a patient, the method comprising:
performing the method of claim 1; and
administering an optimized radiotherapy dose to the patient, wherein the optimized radiotherapy dose is determined by reducing a standard radiotherapy dose by the radiation sensitivity quotient.

13. A method for treating a patient, the method comprising:
obtaining a radiation sensitivity quotient determined by the method of claim 1; and
administering an optimized radiotherapy dose to the patient, wherein the optimized radiotherapy dose is determined by reducing a standard radiotherapy dose by the radiation sensitivity quotient.

14. A method for generating a patient-specific radiotherapy treatment plan, the method comprising:
performing the method of claim 1; and
reducing a standard radiotherapy dose in a reference radiotherapy treatment plan by the radiation sensitivity quotient.

15. A method for generating a patient-specific a radiotherapy treatment plan, the method comprising:
obtaining a radiation sensitivity quotient determined by the method of claim 1; and
reducing a standard radiotherapy dose in a reference radiotherapy treatment plan by the radiation sensitivity quotient.
